(19) United States
(12) Patent Application Publication
(10) Pub. No.: US 2019/0194297 A1
(43) Pub. Date: Jun. 27, 2019

(54) PROCESS TO EXTRACT AND RECOVER KERATIN AND KERATIN ASSOCIATED PROTEIN FROM ANIMAL BODY PARTS

(71) Applicant: TOMORROW WATER, Anaheim, CA (US)

(72) Inventors: KEN TASAKI, REDONDO BEACH, CA (US); LANCE KANEOKA, DIAMOND BAR, CA (US)

(73) Assignee: TOMORROW WATER, Anaheim, CA (US)

(21) Appl. No.: 16/231,194

(22) Filed: Dec. 21, 2018

Related U.S. Application Data
(60) Provisional application No. 62/708,819, filed on Dec. 26, 2017.

(51) Int. Cl. C07K 14/78; C07K 1/34; A61K 8/65; A61L 27/22; A61K 38/01; A61K 47/42; B01D 61/14; B01D 61/02; B01D 61/58; A23K 10/20; A23K 20/147

(52) U.S. Cl. CPC C07K 14/78; C07K 1/34; A61K 8/65; A61L 27/227; A61K 38/015; A23K 20/147; B01D 61/145; B01D 61/025; B01D 61/58; A23K 10/20; A61K 47/42

(57) ABSTRACT

This invention relates to the field of keratin extraction and recovery from keratinous animal body parts (KABP) including, but not limited to, hair, wool, nails, skins, feathers, hooves, claws and other body parts. More specifically, the invention relates to processes to extract keratin from KABP and hydrolyze it to keratin hydrolysates (KHs) using a thermal hydrolysis process (THP) and recover KHs by membrane filtration, in particular, using shear wave-induced ultra- or nano-membrane filtration (SWIUF) or a combination of SWIUF and reverse osmosis (RO), to select given molecular weight (MW) fractions of the protein or KHs or/and to increase the concentration of protein or KHs.

### CROSS-REFERENCE TO RELATED APPLICATION

This application claims the benefit of and priority from U.S. provisional application No. 62/708,819 filed on Dec. 26, 2017 and entitled PROCESS TO EXTRACT AND RECOVER KERATIN AND KERATIN ASSOCIATED PROTEIN FROM ANIMAL BODY PARTS. The contents of the above application are hereby incorporated herein by reference in full.

### FIELD OF INVENTION

This invention relates to the field of keratin extraction and recovery from keratinous animal body parts (KABP) including, but not limited to hair, wool, nails, skins, feathers, hooves, claws and other body parts. More specifically, the invention relates to process to extract keratin from KABP and hydrolyze it to keratin hydrolysates (KHs) using a thermal hydrolysis process (THP) and recover them by membrane filtration, in particular, using shear wave-induced ultrafiltration (SWIUF) or a combination of SWIUF and reverse osmosis (RO) filtration, to select given molecular weight (MW) fractions of the KHs or/and to increase the concentration of KHs.

### BACKGROUND OF THE INVENTION

Keratin and keratin associated protein (KAP) are intracellular proteins in KABP such as hairs, wool, nails, skins, feathers, hooves, claws, and others. The former is hard α-keratins forming microfibrous intermediate filament protein (IFP), while the latter is matrix proteins forming a nonfilamentous matrix. The hard α keratins are highly cross-linked with each other through disulfide bonds, forming a coiled-coil structure with two α-helices making up a protofibril, a bundle of which constitutes a microfibrous IFP which in turn covalently crosslink with KAP. FIG. 1 illustrates the hair structure from a macroscopic level to a molecular level. This complex structure makes the hair structure stable and highly resistant to enzymatic degradation. They are a considerable part of slaughtering wastes brought into rendering plants and mostly disposed of, since they are difficult to hydrolyze due to the stable structure described above, thus having poor digestibility as feed.

About 5 million metric tons (MMT) of wool fibers are wasted worldwide annually during shearing and weaving processes (Zoccola, M.; Aluigi, A.; Patrucco, A., “Microwave-Assisted Chemical-Free Hydrolysis of Wool Keratin,” Text Res., J., 82, 2006 (2012)). The slaughter houses, the rendering industry, and the wool industry in the countries including the U.S., Brazil, and China produce more than 40 MMT of keratin per year (Sharma, S.; Gupta, A., “Sustainable Management of Keratin Waste Biomass: Applications and Future Perspectives,” Braz. Arch. Biol. Technol., 59, January/Dececember, 2016.). KABP are hard to degrade, giving rise to environmental concerns.

Some alternative applications have been developed for these materials such as nitrogen fertilizer for gardening or biodegradable surfactants for fire extinguisher foams (Tonin, C.; Aluigi, A.; Varesano, A.; Vineis, C., “Keratin-Based Nanofibres,” in Nanofibers; Ed. A. Kumar; Intech: Croatia, pp 438 (2010)). However, the size of these markets is limited.

Currently, most keratin wastes from low-valued animal raw materials such as hog hairs and unserviceable wools are simply disposed of at a renderer's cost. New commercial applications of keratin derived from animal wastes for growing markets would bring additional revenue to the rendering industry, which would be also benefited by having less environmental footprints through less landfill disposal.

According to Food Agriculture Organization, over the past 50 years, the global meat production has almost quadrupled from 78 MMT in 1963 to a total of 320 MMT in 2015 (Food and Agriculture Organization of United Nation, “Food Outlook,” June (2016).). The meat consumption is expected to increase to a total of 455 MMT in 2050 (Alexandratos, N.; Bruinsma, J., “World Agriculture Towards 2030/2050,” Food and Agriculture Organization of United Nation, June (2012).). In the meantime, the world population is estimated to be 8.6 billion in 2030 and the middle-class population is growing along with it.(World Population Prospects 2017, United Nation, 2017.). An average adult male and female require 56 and 46 g/day of protein, respectively (“Dietary Reference Intakes for Energy, Carbohydrates, Fiber, Fat, Fatty Acids, Cholesterol, Protein, and Amino Acids, Food and Nutrition Board, Institute of Medicine of the National Academies, the National cademies Press, Washington, D.C., 2002.) On the other hand, finishing pigs require about 140 g/day of protein (“Nutrient Requirements of Swine,” Animal Nutrition Series, National Research Council of the National Academies, National Academies Press, Washington, D.C., 2012.)

KABP consist mostly of keratin protein, up to 94% and the rest are small amounts of lipids and ash (M. B. Esteban, et al. Bioresource Tech., 101, 2472 (2010).). Once extracted from KABP, keratin protein can be hydrolyzed by chemicals, enzyme, or thermal hydrolysis. Protein plays a critical role in the growth and development of young animals. It is in these early stages of life that providing easily digestible sources of protein can improve a long-term growth performance of animals. Hence, the use of hydrolyzed protein as feed is becoming a popular practice among livestock producers.

Free amino acids, the smallest components of a protein, are easier to be digested than a protein itself inside the animal's intestine in general. This is because a protein needs to be first broken down to amino acids before an animal can utilize them for normal metabolic functions. Yet, an increasing number of studies have shown that oligopeptides of protein are just as good as or even better than free amino acids for digestion (Rerat, A.; Simoes, N. C.; Mendy, F.; Roger, L., “Amino Acid Absorption and Production of Pancreatic Hormones in Non-Anesthetized Pigs after Duodenal Infusions of a Milk Enzymatic Hydrolysate or of Free Amino Acids,” Br. J. Nutr. 60, 121 (1988); Webb, K. E.; Matthews, J. C.; DiRenzo, D. B., “Peptide Absorption: A Reviw of Current Concepts and Future Perspectives,” J. Anion. Sci., 70, 3248 (1992); Silk, D. B. A.; Grimble, G. K.; Rees, R. G., “Protein Digestion and Amino Acid and Peptide Absorption,” Proc. Nutr. Soc., 44, 63 (1985); McCalla, J.; Waugh, T.; Lohry, E., “Protein Hydrolysates/Peptides in Animal Nutrition,” in Protein Hydrolysates in Biotechnology, Eds., Pasupulei, V. K.; Demain, Springer, 179 (2010); Stein, H., “The Effect of Including DPS 50RD and DPS EX in the Phase 2 Diests for Weaning Pigs,” South Dakota State University, Brookings S. Dak. (2002).).

For example, Rerat et al. have reported that the rate of absorption of small peptides by animals is higher when compared to the absorption rate of an equivalent amount of free amino acids (Rerat, et al., 1988). Webb et al. have suggested that oligopeptides may have more quantitative importance in ruminant species than do free amino acids (Webb, et al., 1992). Furthermore, Silk et al. have found that peptides with five or fewer amino acid (AA) residues are absorbed with higher efficiency than larger peptides.(Silk, et al., 1985). It seems that a favorable range of MW for digestible protein feeds may be between free AAs and oligopeptides with up to five amino-acid residues. In terms of MW, this range falls between about 75 Da and 1,020 Da. 75 Da is the MW of glycine, the smallest AA, while 1,020 Da is the MW of a pentamer of tryptophan, the largest AA residue, although five consecutive appearances of the tryptophan residue in the keratin protein sequence may be unlikely. Hence, the MW range of between 75 Da and 1,020 Da for KHs may be ideal for feeds. Oligopeptides with MW less than 1,000 Da should have a good digestibility, according to the earlier studies (Rerat et al., 1988; Webb et al., 1992.; Silk, et al., 1985; McCalla, et al., 2010; Stein et al., 2002).

On the other hand, the MW of keratin in animal hair can be as high as 65 KDa or even higher (Nakamura, et al. 2002). Therefore, a degree of hydrolysis on keratin must be high enough to meet this narrow range of the target MWs as set forth herein for keratin hydrolysates when used as feeds.

A keratin-based feed has been already commercialized by Keraplast Technology, LLC by the trade name of OKLP™ at the price of US$220/kg. It is not clear how keratin is extracted from wool, although two processes have been described in the literature: a combination of sulfitolysis and enzymatic process, and the oxidation process (Worth, G.; Kelly, R.; Krsinic, G.; Scott, S.; Roddick-Lanzilotta, A. R. “Topically applied Keratins for Hair and Skin Care,” The Science of Beauty, 5, 53(2015).) According to their Certificate of Analysis, the protein content in the powder is 84.5% and 14% is listed as “residue on ignition” most of which is sodium sulfate, a drying agent, which reduces the protein content in the powder. 90% of wool is composed of keratin in general (Brown, E. M.; Pandya, K.; Taylor, M. M.; Liu, C.-K., “Comparison of Methods for Extraction of Keratin from Waste Wool,” Agricultural Sciences, 7, 670 (2016).).

Recently, KHs are increasingly used for hair, skin, and nail care products. KHs are known to have anti-aging, rejuvenating, and restoration effects on skins and hairs (Mokrejs, P; Hutta, M.; Pavlackova, J.; Egner, P.; Benicek, L., “The Cosmetic and Dermatological Potential of Keratin Hydrolysate,” J. Cosmet. Dermatol., February 6., 12319 (2017).). There is a new category of cosmetics called “cosmetics,” defined as cosmetics that have an active ingredient with physiological effects. The global market size for cosmetics is reported to be $46.93 billion in 2017, expected to reach a value of $80.36 billion by 2023 at a compound annual growth rate (CAGR) of 9.38%. (“Global Cosmeceuticals Market 2018-2023 By Demand, Various Products, Production Cost, Top Regions, Worldwide Consumption & Growth,” Reuters, May 8, 2018.)

The price of hydrolyzed feather meals is about ¢40/kg, while the price of cosmetics with hydrolyzed keratin as a key ingredient varies. For example, the price of Keratin Protein by MakingCosmetics® is US$165/kg of keratin, while that of FK Restore™ by Keraplast Technologies is $(USD)1,785/kg of keratin. MakingCosmetics® claims that its Keratin Protein revitalizes the hair's natural protective layers, rebuilds tensile strength, returns elasticity and reduces breakage. On the other hand, Keraplast Technologies claims that keratin in FK Restore™ binds strongly to severely damaged hair, rebuilding the internal structure and restoring strength, condition, elasticity and shine previously thought to be unrecoverable.

The driving force for cosmetics is two-fold: the increasingly aging society in which a desire to stay looking young has been growing and the tightening regulations on the ingredients used in skin and hair care products have been also encouraging the use of non-toxic keratin ingredients in the products. There are two preferred characteristics for KH as an ingredient for cosmetics: a wide MW distribution from about 500 Da to 100 KDa and a sufficient level of the cysteine residue content. In fact, KHs for cosmetic applications have a wide range of MW, from 500 Da to 125,000 Da, according to Cosmetic Ingredient Review (Bergfeld, W. F.; Belsito, D. V.; Hill, R. A.; Klamino acidssen, C. D.; Liebler, D. C.; Marks, Jr., J. G.; Shank, R. C.; Slaga, T. J.; Snyder, P. W., “Safety Assessment of Keratin and Keratin-Derived Ingredients as Used in Cosmetics,” Cosmetic Ingredient Review, Dec. 10, 2015).

Some KHs have even lower MW. For example, Crotein™ HKP Powder marketed by Croda International is an AA complex, the MW of which is about 150 Da, derived from keratin protein. The company claims that the AA complex penetrates the hair cortex, increasing moisture levels and also plasticity (“Crotein HKP Powder,” CRODA, DS-87, Apr. 25, 1996.)

On the other hand, U.S. Pat. No. 5,679,329A claims KHs with MW up to 200,000 Da as an ingredient for hair care products.(Dupuis, C.; Dubief, C., “Cosmetic Composition for Holding the Hairstyle, Containing a Milk Protein and/or Milk Protein Hydrolysate and a Keratin Hydrolysate,” U.S. Pat. No. 5,679,329A, 1993).

However, most issued patents list KH with the MW of 1,000 Da and less than 50,000 Da for cosmetic applications (Toshioka, et al., U.S. Pat. No. 4,390,525A, 1981.; Vermelho, et al., WO2009000057 A2, 2008; Umeda, et al., US20070128134 A1, 2007; Schrooyen, et al., US20040210039 A1, 2002; Gupta, et al., U.S. Pat. No. 8,575,313 B2, 2013.).

The low MW fraction can penetrate through the cortex to restore the keratin fibrils, while the high MW fraction can cover the surface of hair as coating.

Likewise, for skin care products, it also appears that a combination of KHs with low and high MW fractions gives the ideal outcome since KH acts as a humectant (it binds water from the lower layers of the epidermis to the stratum corneum) as well as an occlusive (it reduces trans-epidermal water loss). Binding water from the lower layers of the epidermis requires low MW fractions to penetrate the epidermis, while the reduction in trans-epidermal water loss is attributable to the higher MW fractions of KH, thereby forming a protective film (Mokrejs, et al., 2017.). The cysteine residue can restore damaged hair by forming the disulfide bonds with its thiol group.

Keratin is also increasingly used as a key material for fabricating scaffolds for tissue engineering. Keratin possesses many distinct advantages over conventional biomolecules, including the biocompatibility, a propensity for self-assembly and intrinsic cellular recognition. The requirement for the MW range of keratin for scaffolds ranges from 40 K to 60 KDa (Verma, V.; Verma, P.; Ray, P.; Ray, A. R., “Preparation of Scaffolds from Human Hair Proteins for Tissue-Engineering Applications,” Biomed Mater., 3, 25007 (2008). Fujii, T.; Takayama, S.; Ito, Y., “A Novel Purification for Keratin-Associated Proteins and Keratin from Human Hair,” J. Biol. Macromol., 13, 92 (2013).).

Conventionally, chemical methods are utilized for extraction of keratin and KAP, typically using chaotropic agents such as urea for swelling the keratin fibrils and also strong oxidizing or reducing agents such as 2-mercaptoethanol, thiols or performig acid for breaking up the disulfide bonds often in the presence of detergent as denaturing agent. (Shavandi, A.; Bekhit, A. A.-D.; Came, A.; Bekhit, A., “Evaluation of Keratin Extraction from Wool by Chemical Methods for Bio-Polymer Application,” J. Bioactive and Compatible Polymers, 1, (2016).). Chemical methods include sulfitolysis, reduction, oxidation, alkali hydrolysis, and the use of ionic liquid (Shavandi, et al., 2016.).

Urea has both a hydrogen donor and acceptor; hence, it interrupts the hydrogen bonds that form the coiled-coil keratin structure, taking the two α-helices apart, thus swelling keratin fibrils for easy access of an oxidizing or a reducing agent. However, a very high concentration of urea is normally required such as 7-8 M which gives rise to high production costs. Reducing the concentration of urea from 7 M to 1 M results in reduced extraction of keratin. (Kakkar, P.; Madhan, B.; Shanmugam, G., “Extraction and Characterization of Keratin from Bovine Hoof: A Potential Material for Biomedical Applications, ” SpringerPlus, 3, 596 (2014).).

One of the most commonly used extraction protocols is called “Shindai Method” (Nakamura, et al., 2002). This protocol involves incubation of the samples at 50° C. for 2 days in a buffer consisting of 25 mM Tris-HCl (pH=8.5), 3M thiourea, 5M urea, and 5% (v/v) 2-mercaptoethanol (2-ME). The recovery yield of protein from the hair samples using the Shindai method was 67% (Nakamura et al., 2002.). In addition, filtration is required for recovery of KH by dialysis which takes up to 6 days. Hence, this process is time-consuming and also costly due to the expensive chemicals.

In addition, according to a study by Kollipara and Zahedi, one fifth of N-termini of proteins and approximately 2% of their Lys residues were shown to be carbamylated during overnight incubation in presence of 2.0M urea at 37° C. Hence, the research group suggested that the usage of urea had to be completely avoided in all sample processing steps (Kollipara, L.; Zahedi, R. P., “Protein Carbamylation: In Vivo Modification or In Vitro Artefact?” Proteomics, 13, 941, (2013)).

Wong, et al. have developed a shorter process without the use of urea while maintaining a similar protein recovery rate (Wong, S. Y.; Lee, C. C.; Ashrafzadeh, A.; Junit, S. M.; Abrahim, N.; Hashim, O. H., “A High-Yield Two-Hour Protocol for Extraction of Human Hair Shaft Proteins,” PLoSONE 11(10): e0164993.). However, many steps were involved in the extraction process, repeating the extraction.

Most of patent applications or issued patents on extraction of keratin from KABP have used either chemical methods or enzymatic methods (Toshioka, I.; Kamimura, Y., “Keratin Hydrolyzate Useful as Hair Fixatives,” U.S. Pat. No. 4,390,525A, 1981.; Vermelho, et al. WO2009000057 A2, 2008; Dupuis, et al., C.; Dubief, C., U.S. Pat. No. 5,679,329A, 1993; Umeda, et al., US20070128134 A1, 2007; Schrooyen, et al., P.; US20040210039 A1, 2002; Gupta, et al., US2012/0130048 A1, 2012.). For chemical processes, using strong oxidizing or reducing agents can degrade the keratin protein structure. Further, the use of such toxic agents gives rise to health and environmental concerns at a large scale production.

Moreover, according to the recent economic analysis of keratin hydrolysis methods by USDA-ARS, an effective keratin hydrolysis by a combination of chaotropic agent and reducing agent is not promising for economic scale up productions primarily due to the high cost of chemicals that are used in these methods (Brown, et al., 2016).

Enzymatic processes of keratin extraction which have been reported mostly entail use of a combination of chemicals (Gousteroval, A.; Braikova, D.; Goshev, I.; Christov, P.; Tishinov, K.; Vasileva-Tonkoval, E.; Haertle, T.; Nedkov, P., “Degradation of Keratin and Collagen Containing Wastes by Newly Isolated Thermoactinomycetes or by Alkaline Hydrolysis,” Letters in Applied Microbiology, 40, 335(2005).; Mokrejs, P.; Krejci, O.; Svoboda, P., “Producing Keratin Hydrolysates from Sheep Wool,” Oriental J. Chem., 27, 1303 (2011); Eslahi, N.; Dadashian, F.; Nejad, N. H., “An Investigation on Keratin Extraction from Wool and Feather Waste by Enzymatic Hydrolysis.,” Prep. Biochem. Biotechnol., 43(7), 624(2013).). The enzymes are generally expensive and the process is time-consuming (Gousteroval, et al., 2005). The extraction yield significantly varies, depending on the original concentration of the KABP. Furthermore, no cysteine was found in the extracted keratin which is disadvantageous for cosmetic applications (Gousteroval, et al.).

Microbial extractions of keratin from KABP have also been reported (Laba, et al., “Biodegradation of Hard Keratins by Two Bacillus Strains,” Jundishapur J. Microbiol. 2014 February; 7(2): e8896.). It was found that naturally occurring bacteria could only extract less than 10% of keratin from animal hair after 4 days of bacteria culture. (Laba, et al.) Others have reported higher extraction rate, ˜70%; yet, it still took 4 days of incubation time (Godbole, S.; Pattan, J.; Gaikwad, S.; Jha, T., “Isolation, Identification and Characterization of Keratin Degrading Microorganisms from Poultry Soil and their Feather Degradation Potential,” International Journal of Environment, Agriculture and Biotechnology, 2 (4), 2060 (2017).). Such a long incubation time can significantly slow down the productivity of keratin extraction.

Ionic liquids have been successfully applied to extract keratin protein with a high extraction yield due to their ability to solvate keratin fibrils (Ji, Y.; Chen, J.; Lv, J.; Li, Z.; Xng, L.; Ding, S., “Extraction of Keratin with Ionic Liquids from Poultry Feather,” Separation and Purification Technology, 132, 577 (2014).). Yet, the high recovery cost of ionic liquids for reuse and their toxicity hinder a wide application of this methodology. Enzymatic hydrolysis is also time-consuming and cost-prohibitive.

Microwave has also been used to extract keratin from wool (Marina Zoccola, Annalisa Aluigi, Alessia Patrucco, Claudia Vineis, Fabrizio Forlini, Paolo Locatelli, Maria Carmela Sacchi and Claudio Tonin, “Microwave-Assisted Chemical-Free Hydrolysis of Wool Keratin,” Bioresource Technology, 70, 111 (1999).). The extraction yield, [W_{KH}]/[W_{KABP}], where W_{KH} and W_{KABP} are the weights of the extracted KH and the original KABP, respectively, varied from 5% to 60%, depending on the temperaure of water in an autoclave inside the microwave and the weight ratio of KABP to water inside the autoclave. The content of either cysteine or cystine in the KH ranged from 0.1 to 1.6, and the MW distribution was between 3K and 8 KDa. There was no high MW fraction in the KH observed by SDS-PAGE.

THP, otherwise known as hot water extraction, has been applied to extract and hydrolyze keratin and KAP from KABP. There are two patent applications based on the same invention using THP to extract keratin from keratinous materials (Fillieres, R.; Belmans, M.; Rogiers, J.; Delmotte, M.; Loussouarn, V., “Method for Producing Highly Digestible Hydrolyzed Keratinaceous Material,” WO2017121897 (A1), 2017; Belmans, M.; Delmotte, M.; Rogiers, J.; Loussouarn, V.; Fillieres, R., “Method for Producing Partially Hydrolyzed Hydrolyzed Keratinaceous Material,” EP3192377 (A1), 2017.). However, the inventions are more focused on the conditions of the post-treatment of keratinous materials after they are treated by THP, or where THP is used as a pretreatment for the process detailed in the claims. The post-treatment involves grinding the THP-processed materials by air turbulence mill in the two inventions. These processes, however, are fundamentally different from the invention described herein in that central to the invention is the use of THP alone without any pre- or post-treatment for extraction and hydrolysis and its conditions to extract keratin from KABP.

There are three previous articles on keratin extraction using THP in the literature (Yin, J.; Rastogi, S.; Terry, A. E.; Popescu, C., “Self-Organization of Oligopeptides Obtained on Dissolution of Feather Keratins in Superheated Water,” Biomacromolecules, 8, 800 (2007); Bhaysar, P.; Zoccola, M.; Patrucco, A.; Montarsolo, A.; Rovero, G.; Tonin, C., “Comparative Study on the Effects of Superheated Water and High Temperature Alkaline Hydrolysis on Wool Keratin,” Textile Res. J., 87, 1696 (2017); Esteban, M. B.; Garcia, A. J.; P. Ramos; Marquez, M. C., “Sub-Critical Water Hydrolysis of Hog Hair for Amino Acid Production,” Bioresource Technology, 101, 2471 (2010).).

Yin, et al. have used THP to hydrolyze feather barbs at temperatures of 180-220° C. and found that feather bars started to dissolve at 180° C. and a complete dissolution of feather barbs was observed at 220° C. for 120 min of reaction time (Yin et al, Biomacromolecules, 8, 800, 2007.). The MWs of the hydrolysates were about 1 K-1.8 KDa extracted and hydrolyzed at 220° C. The MW of the hydrolysates at temperatures lower than 220° C. was not reported. The keratin recovery yield was not reported, nor was the THP condition optimized with respect to the recovery rate and the MW of KHs.

Bhaysar, et al. have reported the extraction of keratin from wool by THP in a combination with 1-5% of alkali agent at 140 or 170° C. for 1 hr (Bhaysar et al., Textile Res. J., 87, 1696 (2017)). They extracted KHs with 3 K-8 KDa of MW with or without the alkali. The keratin recovery yield was not reported, nor was the THP condition optimized with respect to the recovery rate and the MW of KHs.

Esteban, et al. have studied hydrolysis of hog hairs in order to breakdown to AA by THP (Esteban, et al., Bioresource Technology, 101, 2471, 2010). The temperature range for the hydrolysis was 200-300° C. They found that the AA production from hog hairs reached the maximum of 35% with respect to the protein in the original sample at 250° C. It is likely, however, that other hydrolyatste fractions with a range of MWs were dissolved in the effluent, coexisting with the AAs; however, no MW of the dissolved hydrolysates was reported. The keratin recovery yield was not reported, nor was the THP condition optimized with respect to the recovery rate and the MW of KHs.

The previous studies on extraction of keratin by THP do not report the recovery yield of KHs, and neither were the MWs of KHs systematically studied. Despite these studies, the relationship among the THP conditions, the keratin recovery yield, and the MW of KHs, the three characteristics which are crucial for industrial production of KHs, are still unknown. Furthermore, and critically, in the various processes described above, the THP conditions have not been optimized to maximize the recovery yield of KHs from KABP for a given MW fraction.

### BRIEF SUMMARY OF THE INVENTION

The present invention encompasses processes in which KABP including, but not limited to hair, wool, nails, skins, feathers, hooves, claws and other body parts are hydrolyzed by THP and recovered by membrane filtration, specifically SWIUF or a combination of SWIUF or RO, to select given MW fractions of the protein or KHs and/or to increase the concentration of protein or KHs.

Described herein are the optimal conditions to extract and hydrolyze keratin and KAP from KABP in order to maximize the recovery yield for given MW fractions. Moreover, since the functionality of keratin-based cosmetics is sensitive to the MW of the KHs, the optimal conditions for THP, such as temperature and reaction time, can be used to extract keratin or KAP with the desired MW fractions. In the present invention, water is used to extract keratin and KAP from KABP and hydrolyze them, without the use of chemicals.

These and other aspects, embodiments, features, and advantages of the invention will become better understood with regard to the following description, appended claims and accompanying drawings.

Any feature or combination of features described herein are included within the scope of the present invention provided that the features included in any such combination are not mutually inconsistent as will be apparent from the context, this specification, and the knowledge of one of ordinary skill in the art. Additional advantages and aspects of the present invention are apparent in the following detailed description and claims.

### BRIEF DESCRIPTION OF DRAWINGS

The foregoing aspects and the attendant advantages of the present invention will become more readily appreciated by reference to the following detailed description, when taken in conjunction with the accompanying drawings, wherein:

FIG. 1 illustrates the structure of hair in general. The hair includes, but is not limited to, wool, hog hair, human hair, or any other animal hair. a: overlapping cuticle cells; b: cross-section of cuticle; c: cortical cell; d: matrix protein; e: macrofibril; f: microfibril; g: microfilament; h: intermediate filament protein; i: α-helix of keratin protein; j: disulfide bonds between α-helices; k: the coiled coil form; l: the hydrogen bond.

FIG. 2 illustrates a flow chart for an embodiment of a process of the present invention wherein KABP first undergoes THP after which the solid is separated by a screen from the liquid which is filtered by an SWIUF or in a combination of SWIUF and RO to recover and concentrate KHs, respectively. In the figure, a refers to KABP, b is the THP effluent, c is a screen, d and e are the screen effluent and the solid from the screen, respectively, f and g are the permeate and the concentrate from SWIUF, respectively, and h and i are the permeate and the concentrate from RO, respectively. i can be either a keratin-based concentrated solution or further dried to manufacture a keratin-based powder. KABP includes, but is not limited to, animal hairs, wool, nails, skins, feathers, hooves, and claws.

FIG. 3(a) illustrates the transition of keratin α-helix structure to a random coil through denaturing process at temperature=T₁: α-keratin α-helix; b—keratin random coil; c—the hydrogen bond keeping the α-helix structure; R—the side group of the amino acid residue.

FIG. 3(b) illustrates the cleavage of the disulfide bond by H₃O⁺ generated at the temperature=T₂.

FIG. 4 illustrates the keratin recovery yield after THP of hog hairs as a function of the temperature: a—the sample filtered by a vacuum filtration using a 1 μm filter after THP, b—the sample filtered by SWIUF with a 150 KDa membrane after THP, c—the sample heated at 100° C. for 1 hr and subsequently heated at 160° C. for 1 hr, d—the sample preheated at 100° C. for 1 hr and subsequently heated at 180° C. for 1 hr, e—the sample preheated at 100° C. for 1 hr and subsequently heated at 200° C. for 1 hr, and f—the sample preheated at 100° C. for 1 hr and subsequently heated at 220° C. for 1 hr.

FIG. 5 compares the recovery yields of keratin from hog hair between the two-step process described herein, and a one-step process, with the following references: a—the two-step process, b—the one-step process, c—the hog hair was first heated at 100° C. for 1 hr and subsequently heated at 200° C. for 1 hr and filtered by SWIUF with 150 KDa membrane, d—the hog hair was heated at 200° C. for 2 hrs, and e—the hog hair was heated at 200° C. for 1 hr and filtered by SWIUF with 150 KDa membrane.

FIG. 6 shows SDS-PAGE charts for three samples, with the following references: a—FK Restore™ by Keraplast Technologies, LLC, b—Keratin Protein®, and c—the Cyclic Organic Waste Treatment, or COWT® keratin hydrolysate. Sample c was prepared by COWT® at 100° C. for 1 hr followed by heating at 160° C. for 1 hr and filtered by SWIUF with 150 KDa membrane. The numbers shown next to Sample c are the MWs for each band.

FIG. 7(a) shows an MALDI-TOF-Mass spectroscopy chart for FK Restore™ by Keraplast Technologies, LLC, with the following references: a—peaks due to α-cyano-4-hydroxycinnamic acid which is the matrix for MALDI-TOF-Mass spectroscopy and b—keratin oligopeptides.

FIG. 7(b) shows an MALDI-TOF-Mass spectroscopy chart for the COWT® keratin hydrolysate prepared by COWT® at 100° C. for 1 hr followed by heating at 160° C. for 1 hr and filtered by SWIUF with 150 KDa membrane, with the following references: a—peaks due to α-cyano-4-hydroxycinnamic acid which is the matrix for MALDI-TOF-Mass spectroscopy and b—keratin oligopeptides.

FIG. 7(c) shows an MALDI-TOF-Mass spectroscopy chart for Keratin Protein by MakingCosmetics®.

FIG. 8(a) shows an MALDI-TOF-Mass spectroscopy chart for OKLP™ by Keraplast Technologies, LLC, with the following references: a—peaks due to α-cyano-4-hydroxycinnamic acid which is the matrix for MALDI-TOF-Mass spectroscopy and b—keratin oligopeptides.

FIG. 8(b) shows an MALDI-TOF-Mass spectroscopy chart for the COWT® keratin hydrolysate prepared by COWT® at 100° C. for 1 hr followed by heating at 160° C. for 1 hr and filtered by SWIUF with 150 KDa membrane, with the following references: a—peaks due to α-cyano-4-hydroxycinnamic acid which is the matrix for MALDI-TOF-Mass spectroscopy and b—keratin oligopeptides.

FIG. 9 displays SDS-PAGE charts for two samples, with the following references: a—OKLP™ by Keraplast Technologies, LLC and b—the COWT® keratin hydrolysate prepared by COWT® at 100° C. for 1 hr followed by heating at 200° C. for 1 hr and filtered by SWIUF with 150 KDa membrane. The number shown next to Sample b is the MW for the band.

FIG. 10(a) compares the AA composition of the COWT® keratin hydrolysate described in FIG. 9(a) with that of soybean meal (b) on dry matter basis, with the following references: c—essential AAs for monogastoric animals (the bold border line) and d—unessential AAs for monogastric animals (the broken border line). Asx refers to either aspartic or asparagines or both, Glx represents either glutamic acid or glutamine or both.

FIG. 10(b) compares the AA composition of the COWT® keratin hydrolysate described in FIG. 9(a) with that of OKLP™ by Keraplast Technologies, LLC (b) on dry matter basis, with the following references: c—essential AAs for monogastoric animals (the bold border line) and d—unessential AAs for monogastric animals (the broken border line). Asx refers to either aspartic or asparagines or both, Glx represents either glutamic acid or glutamine or both.

FIG. 11 illustrates the process of separating low MW fractions from high MW fractions, using SWIUF with a 10 KDa membrane, with the following references: a—the keratin hydrolysate recovered by SWIUF with a 150 KDa membrane after THP, b—the concentrate, and c—the permeate.

FIG. 12 shows the SDS-PAGE for the COWT® hydrolysate before and after separation using SWIUF with a 10 KDa membrane, with the following references: a—the COWT® hydrolysate before separation, b—the permeate from 10 KDa SWIUF, and c—the concentrate from 10 KDa SWIUF.

### DETAILED DESCRIPTION OF INVENTION

The detailed description set forth below is intended as a description of presently-preferred embodiments of the invention and is not intended to represent the only forms in which the present invention may be constructed and/or utilized. However, it is to be understood that the same or equivalent functions and results may be accomplished by different embodiments that are also intended to be encompassed within the spirit and scope of the invention, and additional variations of the present invention may be devised without departing from the inventive concept. The description itself is not intended to limit the scope of any patent issuing from this description. Rather, the inventors have contemplated that the claimed subject matter might also be embodied in other ways, to include different elements or combinations of elements similar to the ones described in this document, in conjunction with other present or future technologies.

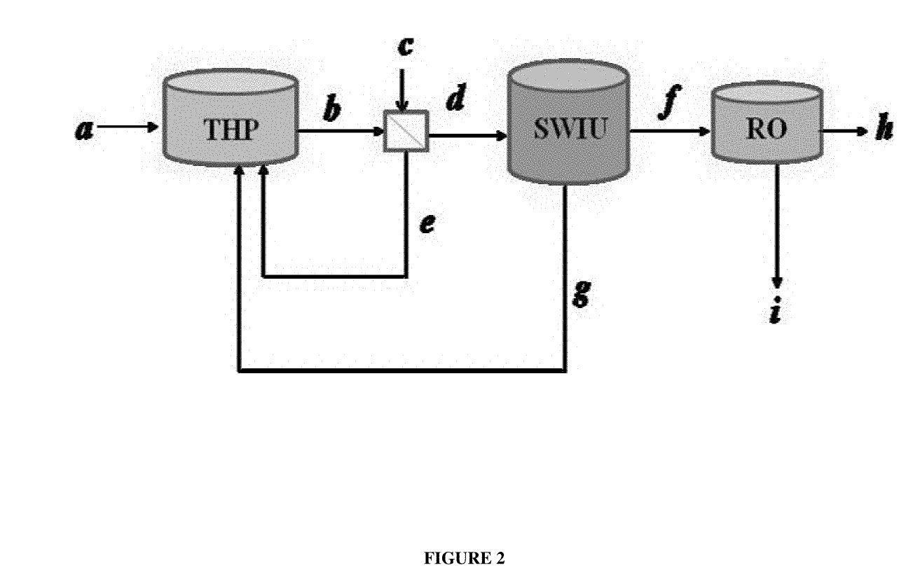

An embodiment of the present invention includes a process in which KABP are hydrolyzed at specific temperatures under pressures to extract keratin and KAP from KABP and recovered by either SWIUF alone or a combination of SWIUF and RO to manufacture keratin-based solutions. Further, the concentrate from RO can be dried by an apparatus including, but not limited to, a freeze drier or a spray drier to manufacture keratin-based powders. The pressure is determined by the saturated vapor pressure at a given temperature. The heating process comprises two steps: preheating at T₁ for a given time followed by heating at T₂ for a given time where T₁<T₂. T₁ can be the denaturing temperature (T_{den}) of keratin protein or the glass transition temperature (T_{g}) of keratin with a given water content, or some other temperatures. T₁ and T₂ can be set prior to the THP reaction through a control panel. The temperature sensor measures the temperature inside the reactor and adjust the energy to heat the reaction vessel in order to maintain the temperature to be either at T₁ or T₂. The reaction vessel can be heated by a heat coil or jacket around outside the vessel or by injecting steam into the vessel. By adjusting the temperatures T₁ and T₂ and the time for THP, keratin can be extracted from KABP and hydrolyzed to KHs with a desirable range of MW fractions and a favorable amount of cysteine residue content. The extracted KHs with the targeted MW fractions can be recovered and concentrated by means of either SWIUF alone or a combination of SWIUF and RO by adjusting the membrane pore size. FIG. 2 illustrates the process to extract keratin from KABP, hydrolyze it, and recover KHs.

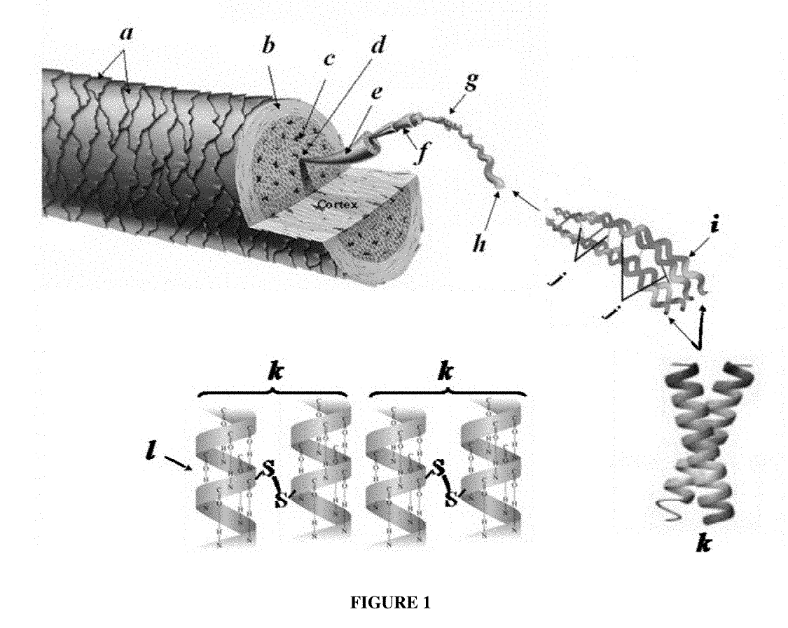

As is shown in FIG. 1, this complex, multilayered structure of animal hair makes the extraction of keratin protein extremely recalcitrant. There are essentially three forces which help form the strong fibril structure: the hydrogen bonds forming the stable α-helical structures, the disulfide bonds connecting the α-helices, and the van der Waals forces between the fibrils which tightly pack layers of fibrils. In the present invention, water is used to extract keratin and KAP from KABP and hydrolyze them, without the use of chemicals.

Water is the most environmentally benign solvent. It is renewable and is a low-cost resource. Water's unique physicochemical properties at high temperatures are well-documented (Plaza, M.; Turner, C., “Pressurized Hot Water Extraction of Bioactives,” Trends in Anal. Chem., 71, 39 (2015).). For example, water is a good solvent for hydrophobic molecules, but a poor solvent for hydrophilic compounds at high temperatures. This is because the dielectric constant (c) of water decreases significantly: it drops by half in going from 20 (ε=78.5) to 200° C. (ε=34.8). It follows that water becomes less polar at high temperatures, starting to dissolve hydrophobic molecules and hence weakening the force which tightly packs bundles of fibrils. At high temperatures, it is likely that water can swell the bundles of fibrils by sneaking into the space between the fibrils, given the reduced dielectric constant of water and therefore, the increased affinity of water to the hydrophobic regions of the protein. The swelling creates essential access for a denaturing agent to break the hydrogen bonds that maintain the α-helices.

Another benefit of a decrease in the dielectric constant by increasing water temperature is that hydrogen bonding becomes less pronounced, destabilizing the α-helical structure. In fact, differential scanning calorimetry (DSC) measurement of wool in water has revealed two peaks around 138 and 144° C., corresponding to the denaturation of α-helix in ortho and para cells which form wool cortex (Wortmann, F.-J.; Deutz, H., “Thermal Analysis of Ortho- and Para-Cortical Cells Isolated from Wool Fibers,” J. Appl. Polym. Sci., 68, 1991 (1998).). That is, at high temperatures around 140′ C., α-helices in the P are likely denatured. This may start happening during the swelling process. Hence, the first step may achieve the swelling of the matrix protein and the fibril bundles and the denaturing of α-helices at the same time. Once the keratin fibril network is swollen and the α-helices denatured, pores or channels are created in the macro or microfibrils for H₃O⁺ to diffuse through the fibril network and also to access the disulfide bonds connecting keratin protein molecules. Hence, T₁ can be T_{den} of keratin.

Moreover, swelling and disentanglement of polymer chains such as peptide chains, which are the first processes prior to dissolution of a polymer, generally become significant above T_{g}. Dissolution of polymers occurs above their T_{g}'s. Thus, if keratin fibrils are heated in water at the temperature above T_{g} of keratin protein molecule, the swelling of the keratin fibrils is particularly enhanced. For example, T_{g} of wool is175° C. However, it has been reported that T_{g} of wool, or any other materials in general, is very sensitive to the water content (Wortmann, F. J.; Rigby, B. J.; Phillips, D. G., “Glass Transition Temperature of Wool as a Function of Regain,” Textile Res. J., 54, 6 (1984)). For example, when the water weight percentage in wool increased up to 20%, T_{g} of wool dropped from 175° C. to as low as 20° C. Hence, the swelling of keratin fibrils can occur below 100° C., depending on the water content. The water content of keratin fibrils in water can increase as the temperature rises, given the increased mobility of water and swelling of the fibrils. Hence, T₁ can be T_{g} of keratin with a given water content.

Accordingly, heating KABP at around T_{g} should facilitate swelling of keratin fibrils prior to extraction of keratin. We refer to this process as “preheating.”

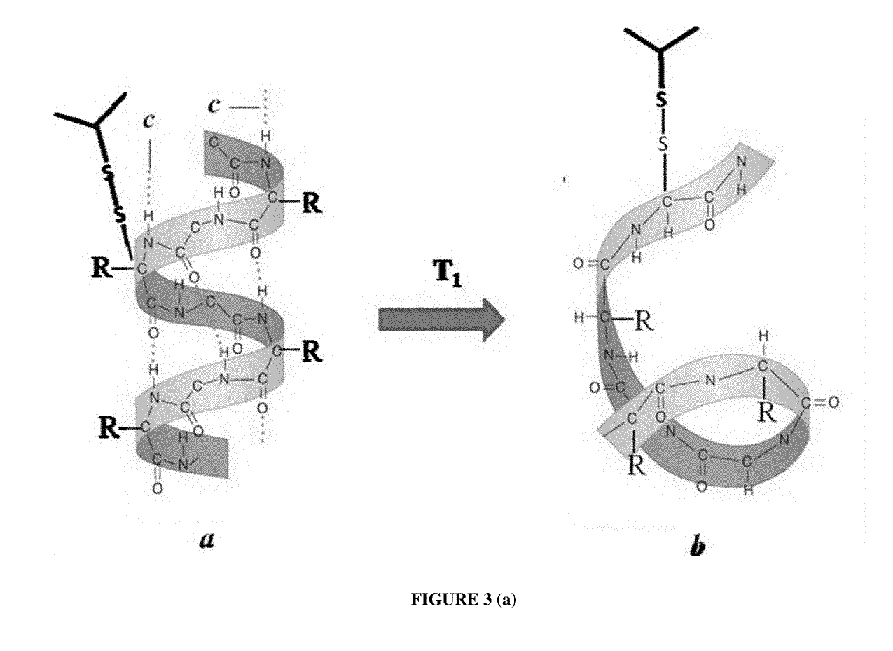

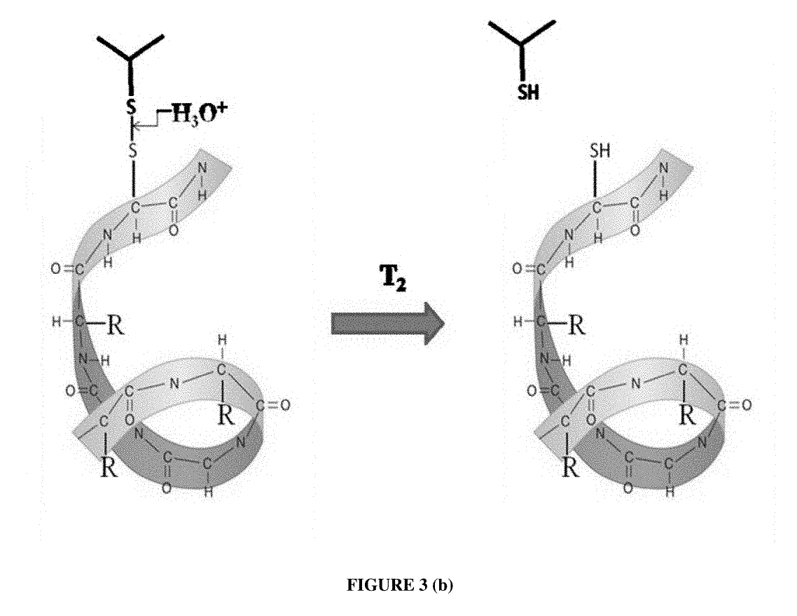

The changes in the dynamic viscosity, the surface tension, and the self-diffusion constant of water at high temperatures also facilitate the wetting and the mass transfer of the components in keratin fibrils. The step described above as a preparation for the extraction of tightly-embedded keratin protein in animal hair has never been performed in the previous THP studies (Esteban, et al., 2010; Yin et al., 2007; Bhaysar, et al., 2016.). FIG. 3(a) illustrates the transition of keratin α-helix structure to a random coil through denaturing, which can occur either at T_{den} or T_{g}. as is described above.

The next step is to cleave the disulfide bonds by H₃O⁺ or OH⁻, now that the IFP is swollen and the α-helices denatured. The water dissociation constant, K_{w}=[H₃O⁺][OH⁻]/[H₂O]², can increase as much as two orders of magnitude in going from 20 to 200° C. (Plaza, et al., 2015). In fact, the concentration of H₃O⁺ and OH⁻ is almost 500 times higher at 200° C. than that at ambient temperature. These ions can break disulfide bonds either as the reducing agent or oxidizing agent.

FIG. 3(b) illustrates the process of the disulfide bond cleavage by H₃O⁺ at the temperature=T₂, described above. Once the disulfide bonds are broken, individual keratin protein molecules are released into water, exposing themselves to water where hydrolysis of the protein molecules takes place under a high concentration of H₃O⁺ at high temperatures. This is the two-step process described herein to increase the extraction yield of keratin from animal hair and help achieve a thorough hydrolysis. This process is termed the “two-step process” to specifically refer to the process by heating KABP twice at different temperatures described herein for extraction of keratin from KABP by THP. In contrast, all other previous THP reports including patents on keratin extraction use a one-step process. An exemplary embodiment of the present invention involves using temperature and the reaction time to control the degree of hydrolysis of keratin protein.

By adjusting the temperatures and the reaction time as described herein, it is possible to have KHs with a desirable range of MW distributions and a favorable amount of the cysteine residue content which is important to restore damaged hair. Further, the relationship between the THP conditions and the MW distribution can be used to control the MW distribution and or the cysteine residue content for given applications. For animal-body parts not consisting of α-keratin such as feathers which are composed of β-keratin or those consisting of other protein such as collagen, a one-step heating process may be sufficient, since these proteins may be easier to extract, given their protein structures.

After hydrolysis, keratin protein is often separated by dialysis which normally takes several days (Deb-Choudhury, S.; Plowman, J. E.; Harland, D. P., “Isolation and Analysis of Keratins and Keratin-Associated Proteins from hair and Wool,” In Methods in Enzymology, Ed. Eichman, B. F.; Elsevier, 568, 279, (2016)). Membrane fouling is also a serious concern in the presence of protein, which results in frequent membrane washings and replacements. The use of SWIUF helps prevent membrane fouling to recover keratin protein more efficiently from the hydrolyzed solution after THP because the vortex flow prevents the build-up of protein on the membrane surface. A combination of SWIUF and RO can also help increase the keratin concentration in the hydrolyzed solution which is typically 2.5% to 6.5% by conventional hydrolysis methods. Such low concentrations require large energy consumption to concentrate the keratin protein solution and then to dry the protein for storage or transportation. For example, using a combination of SWIUF and RO with a 80% recovery rate, the KH concentration can be increased from 10 wt % after THP to as high as 50 wt % at which keratin protein is likely to precipitate, making the separation easier.

In one embodiment, KABP are preheated at T₁ above or equal to T_{den} of keratin for 30 min or longer followed by further heating below or equal to 200° C. for 30 min or longer. Subsequently, SWIUF is used to recover given MW fractions of the KHs and RO to increase the concentration of KHs, if necessary. A dryer including, but not limited to, a spray drier or a freeze drier can be used to form powders from the concentrate from RO.

In another embodiment, KABP are preheated at T₁ above or equal to the T_{g} of keratin with given water content for 30 min or longer followed by further heating below or equal to 200° C. for 30 min or longer. Subsequently, SWIUF is used to recover given MW fractions of the KHs and RO to increase the concentration of KHs, if necessary. A dryer including, but not limited to, a spray drier or a freeze drier can be used to manufacture powders from the concentrate from RO.

In yet another embodiment, KABP are preheated at T₁ above or equal to T_{den} of keratin for 30 min or longer followed by further heating above 200° C. for 30 min or longer. Subsequently, SWIUF is used to recover given MW fractions of the KHs and RO to increase the concentration of KHs, if necessary. A dryer including, but not limited to, a spray drier or a freeze drier can be used to manufacture powders from the concentrate from RO.

In yet another embodiment, KABP are preheated at T₁ above or equal to the T_{g} of keratin with given water content for 30 min or longer followed by further heating above 200° C. for 30 min or longer. Subsequently, SWIUF is used to recover given MW fractions of the KHs and RO to increase the concentration of KHs, if necessary. A dryer including, but not limited to, a spray drier or a freeze drier can be used to manufacture powders from the concentrate from RO.

In yet another embodiment, KABP are preheated at T₁ below T_{g} of keratin with given water content for 30 min or longer followed by further heating below or equal to 200° C. for 30 min or longer. Subsequently, SWIUF is used to recover given MW fractions of the KHs and RO to increase the concentration of KHs, if necessary. A dryer including, but not limited to, a spray drier or a freeze drier can be used to manufacture powders from the concentrate from RO.

In yet another embodiment, KABP are preheated at T₁ below T_{g} of keratin with given water content for 30 min or longer followed by further heating above 200° C. for 30 min or longer. Subsequently, SWIUF is used to recover given MW fractions of the KHs and RO to increase the concentration of KHs, if necessary. A dryer including, but not limited to, a spray drier or a freeze drier can be used to manufacture powders from the concentrate from RO.

In yet another embodiment, KABP are preheated at T₁ below T_{den} of keratin with given water content for 30 min or longer followed by further heating below or equal to 200° C. for 30 min or longer. Subsequently, SWIUF is used to recover given MW fractions of the KHs and RO to increase the concentration of KHs, if necessary. A dryer including, but not limited to, a spray drier or a freeze drier can be used to manufacture powders from the concentrate from RO.

In yet another embodiment, KABP are preheated at T₁ below T_{den} of keratin with given water content for 30 min or longer followed by further heating above 200° C. for 30 min or longer. Subsequently, SWIUF is used to recover given MW fractions of the KHs and RO to increase the concentration of KHs, if necessary. A dryer including, but not limited to, a spray drier or a freeze drier can be used to manufacture powders from the concentrate from RO.

### EXAMPLE 1

Hog hairs collected at a rendering plant were used as the sample for KABP. Hog hairs used for the experiments were cleaned as follows: hog hairs (100 g) were immersed in a solution which was composed of 10 liters of water containing 5% (w/v) of a non-ionic detergent. The solution was stirred for 12 hrs after which the hog hair was removed from the solution. The hog hair was washed by water and then immersed in water only and the solution was stirred for 2 hrs for rinsing after which the hog hair was removed from the solution and placed on an oven at 50° C. overnight. This sample was used for all examples described herein.

A portion of the washed and dried hog hair was set aside for the composition analysis. To prepare a sample, the hair was first cut into small pieces and ground by a pestle. Table 1 lists the composition of the hog hair sample analyzed by the combustion method.

TABLE 1

| Protein (%) | Ash (%) | Lipid (%) | Others (%) |
|---|---|---|---|
| 95.8 | 0.58 | <0.25 | >3.37 |

As is shown in Table 1 above, the hog hair is mostly composed of protein.

The remainder of the hair was used for THP experiments. The THP was performed in a 2 L batch reactor made of stainless steel equipped with a stirrer, and the temperature was controlled by a heat jacket surrounding the reactor and measured by a thermocouple. The pressure applied was the saturated vapor pressure of water at a given temperature. The reactor was sealed during reactions. For each experiment, 10 g of the sample was mixed in 1 L of deionized water and heated at two different temperatures for a given time consecutively. Then, the solution after reaction was filtered by SWIUF with a 150 KDa membrane to remove small fragments of unreacted hair.

Separately, the solution after reaction was filtered by microfiltration using a 1 μm filter. The sample collected from the permeate of the filtration was used to measure the protein concentration and the MW fractions. The protein concentration was determined by hydrolyzing the sample and analyzing the AA by HPLC, while the MW was measured by both MALDI-TOF mass spectroscopy and SDS-PAGE. The keratin recovery yield was calculated by the following equation:

Recovery Yield=[Wₚ^{s}]/[Wₚ^{o}]

where Wₚ^{s} and Wₚ^{o} refer to the weight of KHs in the permeate from SWIUF and the weight of keratin protein in the original sample, respectively. Both Wₚ^{s} and Wₚ^{o} were measured by HPLC after hydrolysis of either KHs or keratin protein to AAs

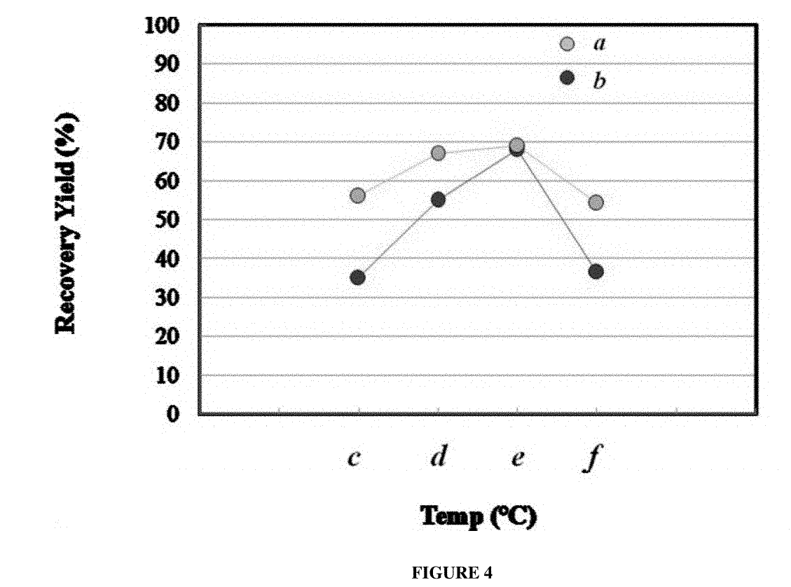

FIG. 4 displays the recovery yield as a function of the reaction condition: a and b refer to the sample filtered by microfiltration and ultrafiltration, respectively. THP was run under different conditions: heating the sample at T₁ for 1 hr after heating at T₂ for 1 hr, where T₁=100° C. and T₂=160° C. (c); T₁=100° C. and T₂=180° C. (d); T₁=100° C. and T₂=200° C. (e); T₁=100° C. and T₂=220° C. (f). It is clear from FIG. 4 that the microfiltration overestimates the protein in the sample after THP, hence the higher recovery yields for the sample by microfiltration than that by ultrafiltration except for Experiment e. It is likely that tiny unreacted hair fragments in the sample smaller than 1 μm passed through the 1 μm filter and stayed in the filtrate. These hair fragments underwent a rigorous hydrolysis during the AA analysis, becoming AAs and picked up by HPLC which has led to the overestimation. Hence, it is important to perform an ultrafiltration using a 150 KDa or possibly a 200 KDa membrane, after THP to remove unreacted hair fragments prior to characterization of the keratin hydrolysate.

FIG. 4 demonstrates that when T₂=200° C. (e), the recovery yield achieved was almost 70%, and that when the temperature goes beyond 200° C., the recovery yield decreases. A study has been compiled on the keratin recovery yields by chemical processes with the finding that the recovery yield ranged from 5 to 53% (Shavandi, A.; Bekhit, A. E.-D.; Came, A; Bekhit, A., “Evaluation of keratin extraction from wool by chemical methods for bio-polymer application,” J. Bioactive Compatible Polymers, 1-15, 2016.). A high recovery yield of 65% using a chemical process has been also reported (Fujii, T.; Takayama, S.; Ito, Y., “A Novel Purification for Keratin-Associated Proteins and Keratin from Human Hair,” J. Biol. Macromol., 13, 92 (2013)). In contrast, a 70% recovery yield in an embodiment of the present invention is high compared to the studies and what has been described in the literature.

### EXAMPLE 2

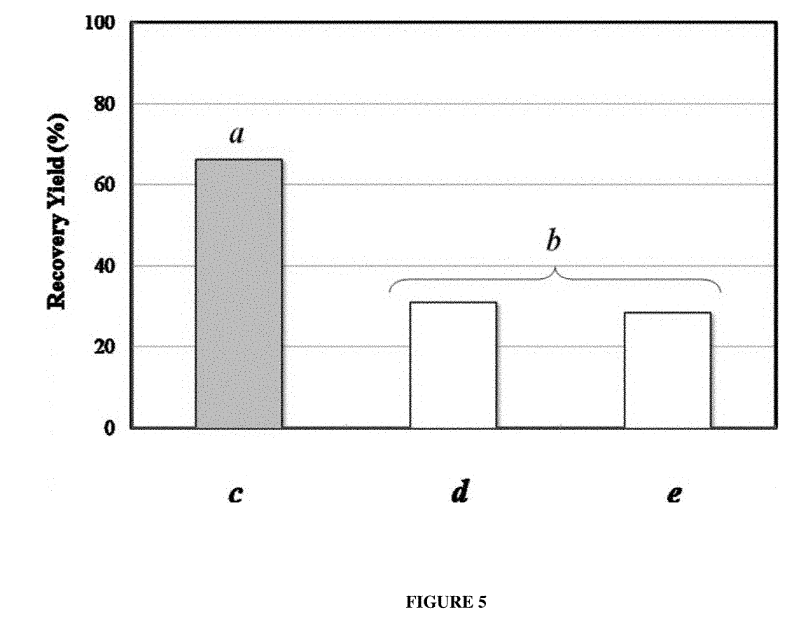

FIG. 5 compares the recovery yield between the two-step process and the one-step process described herein. The recovery yield of the one-step process, either d or c, is approximately half the recovery yield of the two-step process. This further demonstrates the effectiveness of the two-step process as described herein.

### EXAMPLE 3

Two key characteristics are at least required for keratin used in cosmetics: a wide range of MW distribution and the adequate cysteine residue content which is used to cross-link between two α-helices through disulfide bonds for hair restoration. Two sample are chosen herein from the market for a comparison with the keratin hydrolysate of the present invention: one is FK Restore™ by Keraplast Technologies, LLC and Keratin Protein by MakingCosmetics®, both of which are already commercially available as keratin-based hair-care products. Keraplast Technologies is known to use at least two processes: sulfitolysis followed by an enzymatic process and an acid oxidation process to extract keratin from wool (Worth, et al., 2015.). Their products are formulated by mixing keratin hydrolysates from various processes. MakingCosmetics® uses an alkali pretreatment prior to an enzymatic process to extract keratin from wool (https://www.makingcosmetics.com/Keratin-Protein-Hydrolyzed_p_924.html). Neither manufacturer uses THP for keratin extraction. A comparison is made between the MW distribution and the cysteine residue content of the keratin hydrolystate product of the present invention against the keratin hydrolystate product of these manufacturers, which is discussed below.

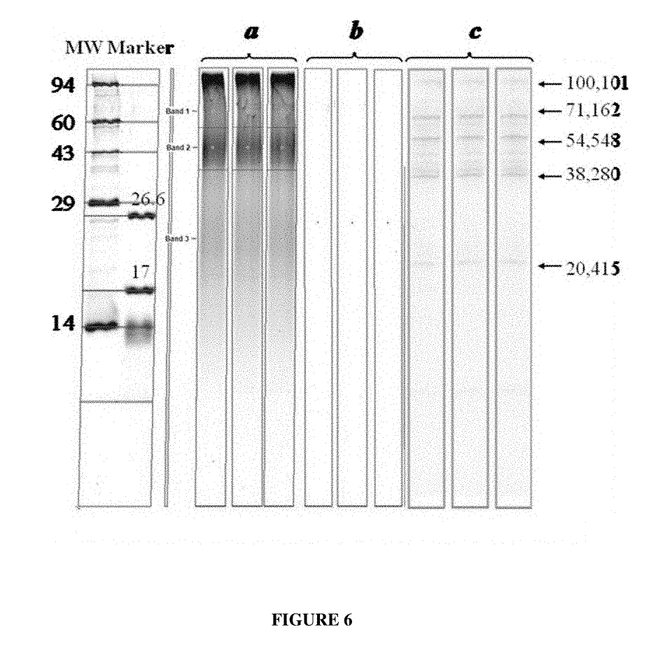

FIG. 6 displays the SDS-PAGE patterns for the three samples: FK Restore™ (a), MakingCosmetics® (b), and our keratin hydrolysate extracted by COWT® using the two-step process and filtered by SWIUF with a 150 KDa membrane (c). The last keratin hydrolysate (c) will be referred to as the COWT® hydrolysate. The condition to obtain the COWT® hydrolysate was T₁=100° C. and T₂=160° C. for 1 hr at each heating step. The three samples were subjected to triplicate measurements. FK Restore™ exhibits a continuous smear from the top to the bottom, showing a wide range of MW fraction from around 10 KDa to over 100 KDa, while there is no band appearing in the sample of MakingCosmetics®, which means that there is no high MW fraction for this product. The COWT® hydrolysate, on the other hand, shows a number of bands, some dark and others light, from 20 KDa up to over 100 KDa, which are similar to those obtained by a chemical process (Fujii, et al. (2013); Yamauchi, et al., (1998)). Hence, the COWT® hydrolysate also has a wide range of MW distribution. SDS-PAGE has a limitation in measuring MW fractions lower than around 10 KDa. For those with MW below 10 KDa, a different instrumentation is required to probe such as mass spectroscopy. MALDI-TOF-Mass spectroscopy was used herein, which is described below.

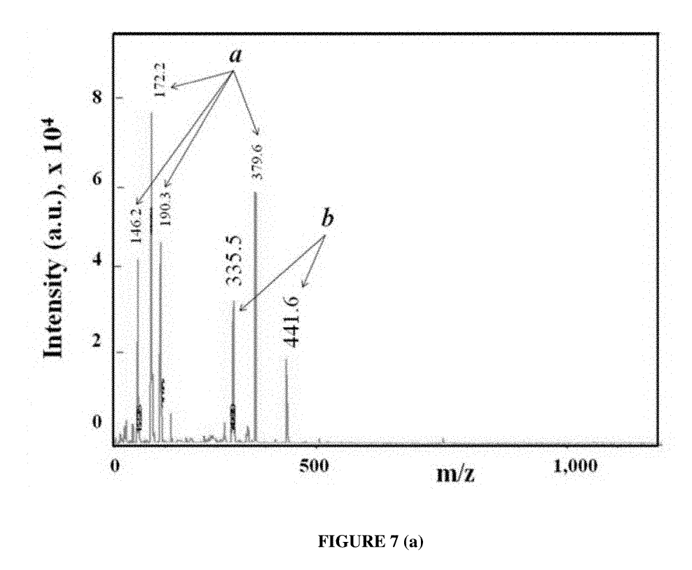

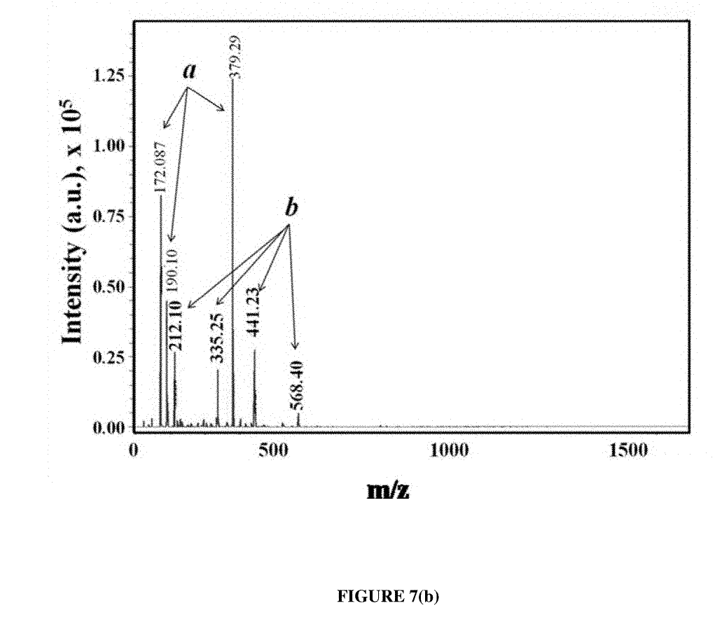

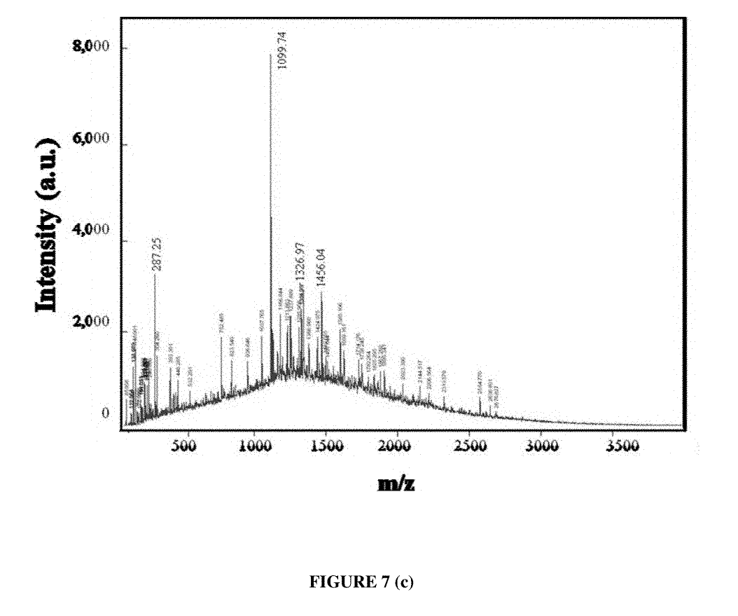

FIG. 7 illustrates the MALDI-TOF-Mass spectra of FK Restore™ (a), the COWT® hydrolysate (b), and MakingCosmetics® (c). The reaction condition for the COWT® hydrolysate was the same as those shown in FIG. 6. The peaks depicted as a in both FIGS. 7(a) and (b) are due to α-cyano-4-hydroxycinnamic acid which is a matrix used for MALDI-TOF-Mass spectroscopy; hence, these peaks are artifact.

For FK Restore™, no significant peaks appear above 500 Da below which there are only two peaks. On the other hand, there are much more peaks in the spectrum of the COWT® hydrolysate. Furthermore, the intensity, which indicates the concentration of each peak, is an order of magnitude higher than that for FK Restore™, increased from 10⁴ to 10⁵. This suggests that COWT® hydrolysate has more low MW fractions than FK Restore™.

As to MakingCosmetics®, there are many more peaks lower than 3,000 Da. Combined with the results from FIG. 6, it is found that this product has fractions with only MWs lower than 3,000 Da with no high MW fractions. In conclusion, the COWT® hydrolysate has a wide range of MW fractions from a few hundred Da up to 100 KDa with more low MW fractions than FK Restore™.

The cysteine residue content was measured by the AA analysis for the three samples. Table 2 below summarizes the results. The existing products FK Restore™ and MakingCosmetics® have the cysteine content of 7.5% and 1.6%, respectively. In general, the higher the cysteine content the keratin hydrolysate has, the better its performance becomes. Hence, FK Restore™ may be considered a high-end product, while MakingCosmetics® may be considered either a mid-end or a low-end product, given the cysteine content. Though the cysteine content of the COWT® hydrolysate is sensitive to the reaction temperature, it can be 2.9% which is between the cysteine content of FK Restore™ and MakingCosmetics®.

TABLE 2

| | COWT ® | | FK | |
|---|---|---|---|---|
| | hydrolysate | | Restore ™ | MakingCosmetics ® |
| T₁, ° C.^{a} | 100 | 100 | | |
| T₂, ° C.^{a} | 160 | 200 | | |
| Cysteine, % | 2.9 | 0.6 | 7.5 | 1.6 |

^{a}The reaction time was 1 hr.

Though it is not clear how high the cysteine content should be to be effective as a hair-care product, Table 2 indicates that the cysteine content of the COWT® hydrolysate can be controlled by the reaction condition to some extent.

### EXAMPLE 3

We characterized the COWT® hydrolystate for feed applications. We use OKLP™ by Keraplast Technologies, which is already in the market, for comparison of the characteristics.

Silk et al. have reported that peptides with five or fewer AA residues are absorbed with higher efficiency than larger peptides (Silk, et al., 1985). It appears that a favorable range of MW for digestible protein feeds may be between free AAs and peptides with five amino-acid residues. In terms of MW, this range falls between about 75 Da and 1,020 Da. 75 Da is the MW of glycine, the smallest AA, while 1,020 Da is the MW of a pentamer of tryptophan, the largest AA residue. On the other hand, the MW of keratin in animal hair can be as high as 65 KDa or higher (Nakamura, et al., 2002). The higher the MW of protein hydrolysates, the more difficult it is to digest by an animal in general.

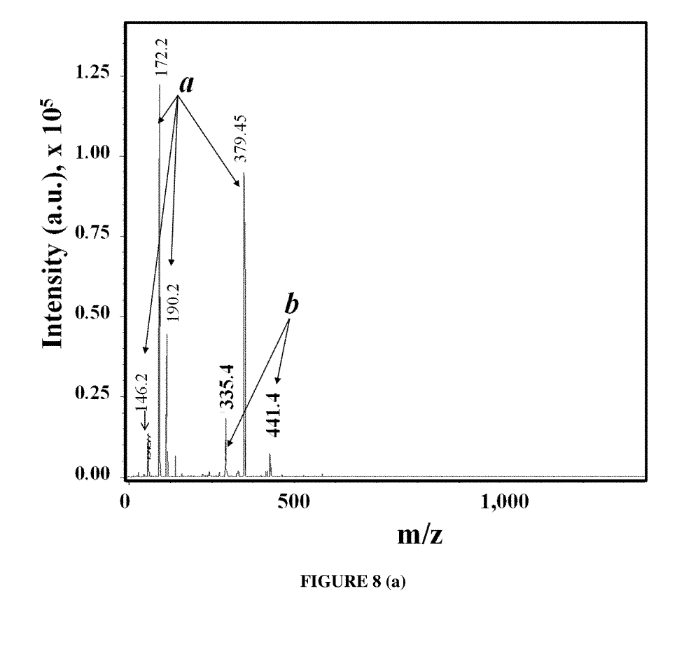

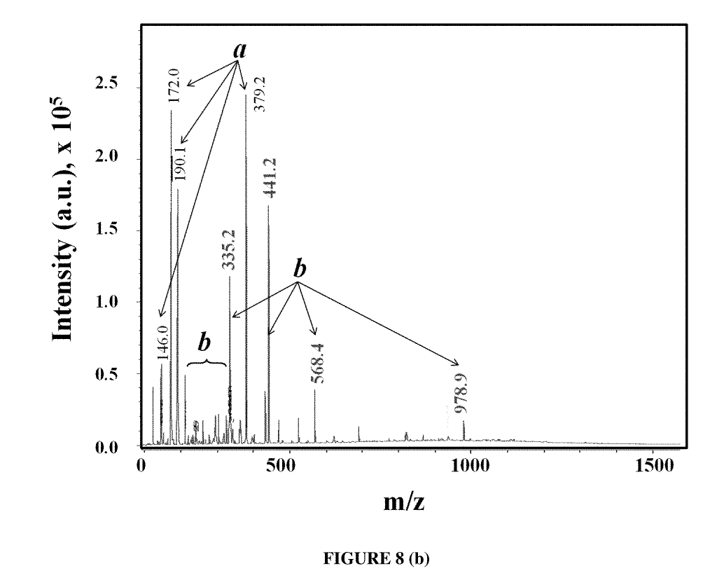

FIG. 8(a) displays the MALDI-TOF-Mass spectroscopy for OKLP™ by Keraplast Technologies. No visible peak above 1,000 m/z was observed; hence, those below 500 m/z are shown. As is the case in FIG. 7(a), the peaks designated as a are due to α-cyano-4-hydroxycinnamic acid, which has nothing to do with keratin. There are only two relatively small peaks at 335.4 and 441.4 which can be assigned to oligopeptides of keratin hydrolysates.

FIG. 8(b) shows the results for the COWT® hydrolystate. The condition to obtain the COWT® hydrolysate was T₁=100° C. and T₂=200° C. for 1 hr at each heating step. Significantly more peaks are observed up to 1,000 m/z, compared to those for OKPL™. It is apparent that the COWT® hydrolystate has more low MW fractions, compared to OKPL™. Low MW fractions are important for feeds since they are more easily absorbed by animals.

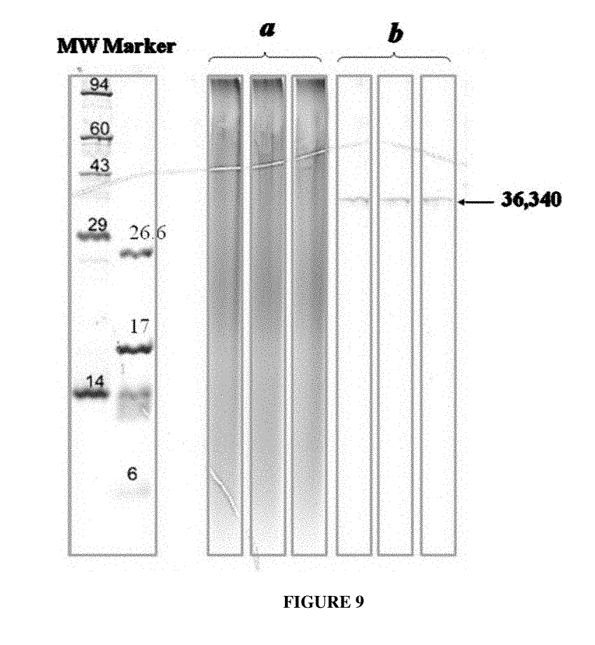

FIG. 9 shows the SDS-PAGE for OKLP™ and the COWT® hydrolystate prepared under the same conditions as that shown in FIG. 8(b). Each sample was subjected to triplicate measurements. OKLP™ (a) exhibits a long smear from the top to the bottom. Combined with the result from FIG. 8, OKLP™ is mainly composed of mid to high MW fractions from several thousand Da to over 100 KDa. On the other hand, the COWT® hydrolystate (b) has only one band at 36,340 Da. Combined with the result from FIG. 7(b), it can be concluded that the COWT® hydrolystate has a mixture of low and high MW fractions. Compared to OKLP™, the COWT® hydrolystate has less high MW fractions which may be favorable as feeds.

The AA composition is an important characteristic for feeds. Especially for monogastric animals such as hogs and poultry, essential AAs need to be adequately included in the feed. The bars with the dark color and the bold border line indicate the essential AAs for hogs.

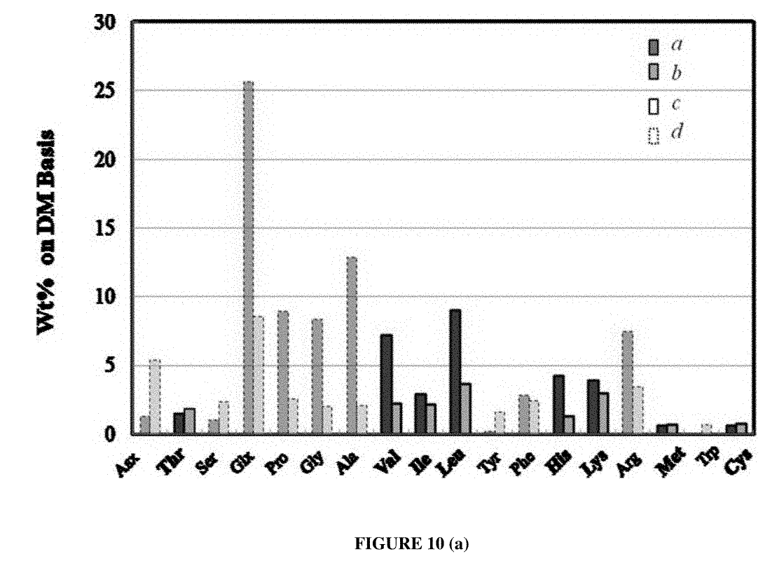

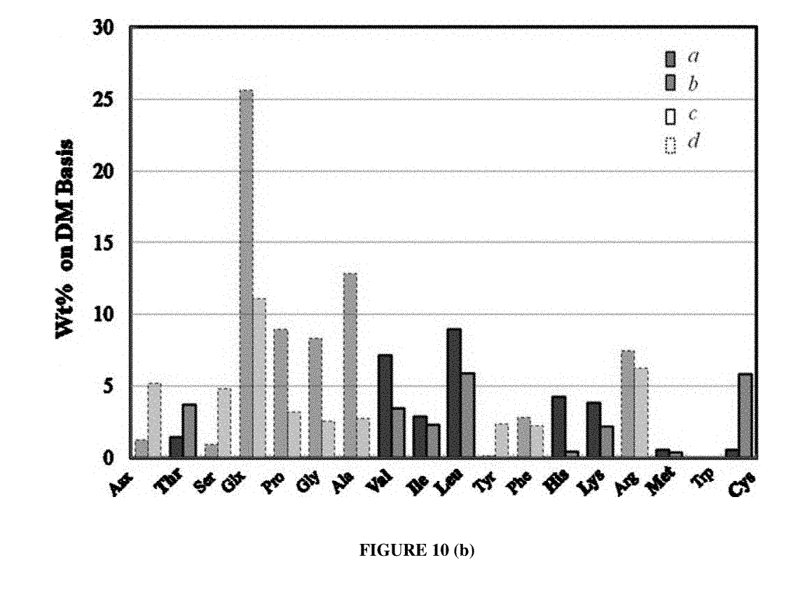

FIG. 10(a) compares the AA composition between that of the COWT® hydrolystate prepared under the same conditions as shown in FIG. 8(b) and that of soybean meal on dry matter basis. Soybean meal is a popular feed for livestock, especially for hogs and poultry. Though soybean meal has slightly more threonine and cysteine than the COWT® hydrolystate, the COWT® hydrolystate has more valine, isoleucine, leucine, histidine,and lysine than soybean meal. Some, such as valine and leucine, are especially higher in content than soybean meal which suggests the COWT® hydrolystate is a favorable feed for hog and poultry. In total, the COWT® hydrolystate has about 14.8% more essential AAs than OKLPTM

FIG. 10(b) compares the AA composition between that of the COWT® hydrolystate prepared under the same conditions as shown in FIG. 8 (b) and that of OKLP™ on dry matter basis. Though OKLP™ has more threonine and significantly more cysteine than the COWT® hydrolystate, the COWT® hydrolystate has more valine, isoleucine, leucine, histidine, and lysine than OKLP™. In total, the COWT® hydrolystate has about 5% more essential AAs than OKLP™.

Table 3 below lists the composition of the COWT® hydrolystate powder and OKLP™. The COWT® hydrolystate powder was prepared by first concentrating the permeate from FMX® filtration system by reverse osmosis and drying the concentrated solution by a vacuum evaporator. The thick slurry from the vacuum evaporator was placed in an oven at 60° C. overnight and the solid was ground to powder form by use of a pestle.

TABLE 3

| Sample | Protein (%) | Ash (%) | Fat (%) | Water (%) | Others (%) |
|---|---|---|---|---|---|
| COWT ® | 89.2 | 6.1 | <0.5 | 4.6 | 3.37 |
| hydrolystate | | | | | |
| powder | | | | | |
| OKLP ™ ^{a} | 84.5 | — | — | 1.7 | 14.6^{b} |

^{a} Based on the certificate of analysis provided by Keraplast Technologies, LLC.
^{b}Mostly Na₂SO₄ based on the information provided by Keraplast Technologies, LLC.

Na₂SO₄ was used as a drying agent for OKLP™. Since a drying agent was not used, the protein content of the COWT® hydrolystate powder is higher than that of OKLP™. Most of the data in Table 3 was obtained through a combustion method, except for H₂O, which was measured by the sample weight difference before and after using the Speed-Vac®.

### EXAMPLE 4

Recovering a particular MW fraction from a wide range MW distribution of the keratin hydrolysate is a useful tool for a variety of applications. This can be accomplished by adjusting the pore size of the SWIUF membrane. Changing the condition for the COWT® can also control the MW distribution to some extent. However, adjusting the membrane pore size can provide much more flexibility for separating an MW fraction from others. For example, a material used for scaffold fabrication requires high MW fractions in general and in the case of keratin, the MW of 40 KDa˜60 KDa is desirable in particular (Kakkar, P.; Madhan, B.; Shanmugam, G., “Extraction and Characterization of Keratin from Bovine Hoof: a Potential Material for Biomedical Applications, “SpringerPlus, 3, 596 (2014); Deb-Choudhury, et al., 2016.)).

However, as has been shown in FIGS. 6, 8(b), and 9, COWT® hydrolystates have a wide range of MW distribution including fractions with MW lower than 1,000 Da. In this example, we demonstrate the separation of the low MW fractions lower than 10 KDa from higher MW fractions.

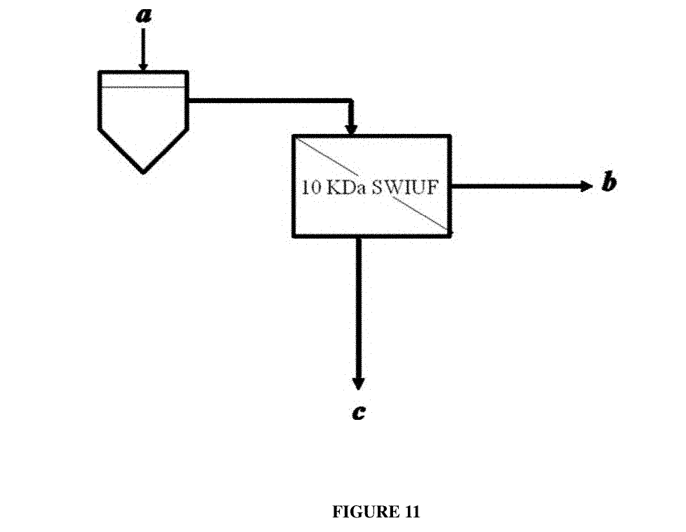

FIG. 11 illustrates the process of separating low MW fractions from high MW fractions of the COWT® hydrolysate, using SWIUF with a 10 KDa membrane. In theory, fractions with MW lower than 10 KDa should be collected in the permeate, while those with MW higher than 10 KDa should stay in the concentrate. However, in reality (or in practice), it is rare to completely separate fractions exactly at 10 KDa when membrane filtration is used. Some fractions slightly beyond or below 10 KDa can be in either the permeate or the concentrate from the filtration.

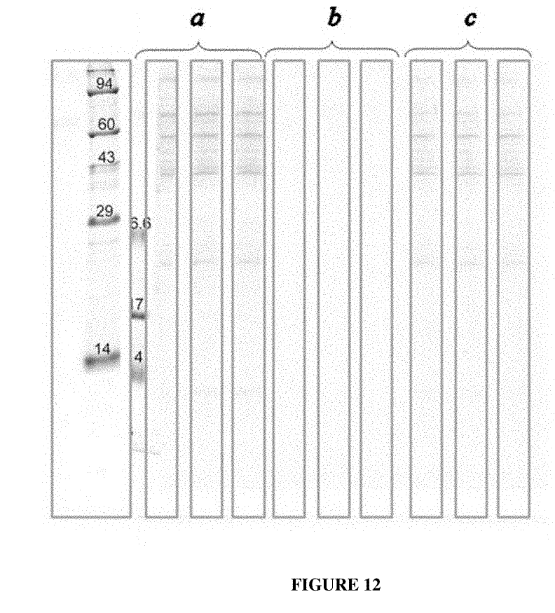

FIG. 12 displays the SDS-PAGE for the COWT® hydrolystate prepared under the same conditions as shown in FIG. 8(b) and subjected to SWIUF with a 10 KDa membrane (a), the permeate from the 10 KDa separation (b), and the concentrate from the 10 KDa separation (c). It is clearly shown that the permeate shows no bands, indicating no high MW fractions in the permeate, while the concentrate maintains the same band patterns as those before the separation, demonstrating the separation of the low MW fraction from the high MW fractions.

Various modifications of the invention, in addition to those described herein, will be apparent to those skilled in the art from the foregoing description. Such modifications are also intended to fall within the scope of the appended claims. Each reference cited in the present application is incorporated herein by reference in its entirety.

Although there has been shown and described the preferred embodiment of the present invention, it will be readily apparent to those skilled in the art that modifications may be made thereto which do not exceed the scope of the appended claims. Therefore, the scope of the invention is only to be defined by the following claims.

What is claimed is:

1. A method to extract and hydrolyze keratin and keratin associated protein from keratinous animal body parts using a two-step thermal hydrolysis process, wherein said method comprises:
placing one or more keratinous animal body part in a reactor;
immersing said body part in said reactor in water;
preheating said body part at T₁ which is ≥T_{g} of keratin with a given water content under a saturated water vapor pressure at T₁ for at least 10 minutes;
heating further said body part at T₂ which is ≤200° C. for at least 10 minutes;
wherein after said two-step heating process, effluent from said reactor is filtered using shear wave-induced ultrafiltration (SWIUF); and
wherein said filtration is used to recover desired molecular weight (MW) fractions of keratin hydrolysates (KHs) or to increase the concentration of recovered KHs.

2. The method of claim 1, wherein said effluent is filtered using a combination of SWIUF and reverse osmosis (RO) filtration.

3. The method of claim 1 or 2, wherein said T₂ is ≥200° C.

4. The method of claim 1 wherein T₁ is ≥T_{den} of the said keratin protein in the said body part.

5. The method of claim 1 or 6 wherein concentrate from said SWIUF is returned to said reactor for further reaction to increase the recovery yield.

6. The method of claim 1 or 6 further comprising the step of returning solid from said body part that was screened to said reactor for further reaction to increase the recovery yield.

7. The method of claim 2 wherein said RO has a membrane that has a rejection rate that is within the range of 80% and 99%.

8. The method of claim 2 wherein concentrate from said RO is dried to manufacture a powder using an apparatus that is selected from the group consisting of a freeze dryer and spray dryer.

9. A method to extract and hydrolyze keratin and keratin associated protein from keratinous animal body parts using a thermal hydrolysis process, wherein said method comprises:
placing one or more keratinous animal body part in a reactor;
immersing said body part in said reactor in water;
wherein said body part is heated at a temperature that is ≥T_{den} or T_{g} of keratin for at least 1 hour;
wherein after said heating, filtration selected from the group consisting of shear wave-induced ultrafiltration (SWIUF) or a combination of SWIUF and reverse osmosis (RO) is used to recover given molecular weight (MW) fractions of keratin hydrolysates (KHs) to increase the concentration of KHs or to dry the RO concentrate to make the KHs in powder form.

10. The method of claim 9 wherein the RO concentrate is dried by an apparatus selected from the group consisting of a spray dryer and a freeze dryer.

11. The method of claim 1 wherein said body part is heated only once at a temperature that is ≥T_{g} or T_{den}.

12. The method of claim 1 wherein said process is used to produce KHs for cosmetic applications.

13. The method of claim 1 wherein said process is used to produce KHs for animal feed.

14. The method of claim 1 wherein said process is used to produce KHs for animal nutrition supplements.

15. The method of claim 1 wherein said process is used to produce KHs for biomedical applications.

16. The method of claim 1 wherein steam is injected into the reactor prior to heating.

17. The method of claim 15 wherein said biomedical application is production of a KH-based material to fabricate scaffolds for tissue engineering.

18. The method of claim 15 wherein said biomedical application is production of a KH-based wound-healing agent.

19. The method of claim 15 wherein said biomedical application is production of a KH-based drug carrier.

20. A method to extract and hydrolyze keratin and keratin associated protein from keratinous animal body parts using a two-step thermal hydrolysis process, wherein said method comprises:
placing one or more keratinous animal body part in a reactor;
injecting steam into said reactor;
preheating said body part at T₁ which is ≥T_{g} of keratin with a given water content under a saturated water vapor pressure at T₁ for at least 10 minutes;
heating further said body part at T₂ which is ≤200° C. for at least 10 minutes;
wherein after said two-step heating process, effluent from said reactor is filtered using filtration selected from the group consisting of shear wave-induced ultrafiltration (SWIUF) or a combination of SWIUF and reverse osmosis (RO) is used to recover given molecular weight (MW) fractions of keratin hydrolysates (KHs) to increase the concentration of KHs or to dry the RO concentrate to make the KHs in powder form.
